(19) United States
(12) Patent Application Publication
(10) Pub. No.: US 2020/0347338 A1
(43) Pub. Date: Nov. 5, 2020

(54) MULTI SENSOR FOR A BIOREACTOR, BIOREACTOR, METHOD FOR PRODUCING A MULTI SENSOR, AND FOR MEASURING PARAMETERS

(71) Applicant: EPPENDORF AG, Hamburg (DE)

(72) Inventors: Sebastian Selzer, Aachen (DE); Rudolf Petkau, Aachen (DE); Guido Ertel, Dormagen (DE); Wolfgang Streule, Titz-Rödingen (DE)

(73) Assignee: EPPENDORF AG, Hamburg (DE)

(21) Appl. No.: 16/960,928

(22) PCT Filed: Dec. 18, 2018
(86) PCT No.: PCT/EP2018/085573

§ 371 Date: Jul. 9, 2020

(30) Foreign Application Priority Data
Jan. 17, 2018 (EP) 18152153.5

(51) Int. Cl. C12M 1/34; C12M 1/00; C12M 1/21

(52) U.S. Cl. CPC C12M 41/12; C12M 23/28; C12M 41/02; C12M 41/44; C12M 41/28; C12M 41/30; C12M 41/26

(57) ABSTRACT

A multisensor and a process for the production of the multisensor for a bioreactor for use in cell culture and/or in microbiology is disclosed. The multisensor comprises at least three measurement arrangements configured to measure at least three parameters, where a first of the three measurement arrangements is configured to carry out an impedance measurement and/or a capacitive measurement, and where the first measurement arrangement has at least two electrodes which comprise an electrically conductive plasti.

### CROSS-REFERENCE TO FOREIGN PRIORITY APPLICATION

The present application claims the benefit under 35 U.S.C. §§ 119(b), 119(e), 120, and/or 365(c) of PCT/EP2018/085573 filed Dec. 18, 2018, which claims priority to European Application No. 18152153.5 filed Jan. 17, 2018.

### FIELD OF THE INVENTION

The invention relates to a multisensor for a bioreactor for use in cell culture and/or in microbiology, to a bioreactor for use in cell culture and/or in microbiology, to a process for the production of a multisensor, and to a process for the measurement of parameters in a bioreactor for use in cell culture and/or in microbiology.

### BACKGROUND OF THE INVENTION

Bioreactors, sensors, and processes for the measurement of parameters are known by way of example from EP 2725095 B1, WO 2016092281 A1, CN 105044038 A. However, further improvements are desirable, in particular, for use in cell culture and/or in microbiology.

### SUMMARY OF THE INVENTION

It is therefore an object of the present invention to provide an improved multisensor for a bioreactor for use in cell culture and/or in microbiology, an improved bioreactor for use in cell culture and/or in microbiology, an improved process for the production of a multisensor, and an improved process for the measurement of parameters in a bioreactor for use in cell culture and/or microbiology.

In particular, it is an object of the present invention to provide a low-cost bioreactor for use in cell culture and/or in microbiology, a low-cost process for the production of a multisensor and a low-cost process for the measurement of parameters in a bioreactor for use in cell culture and/or in microbiology. It is, in particular, also an object of the present invention to provide the following which are also suitable for small operating volumes: a bioreactor for use in cell culture and/or in microbiology, a process for the production of a multisensor, and a process for the measurement of parameters in a bioreactor for use in cell culture and/or in microbiology.

This object is achieved in accordance with the invention via a multisensor for a bioreactor for use in cell culture and/or in microbiology, where the multisensor comprises at least three measurement arrangements configured to measure at least three parameters, where a first of the three measurement arrangements is configured to carry out an impedance measurement and/or a capacitive measurement, and where the first measurement arrangement has at least two electrodes which comprise an electrically conductive plastic or consist thereof.

The multisensor described here is therefore configured by means of the at least three measurement arrangements to measure at least three parameters, and it is preferable here that at least three different measurement arrangements are used and/or that at least three different parameters can be measured. The first of the three measurement arrangements is suitable for carrying out an impedance measurement and/or a capacitive measurement. To this end, the first measurement arrangement has two or more electrodes which comprise an electrically conductive plastic or consist thereof.

The invention is based inter alia on the discovery that, specifically for the use in cell culture and/or in microbiology, there is frequently only little space available for the arrangement of sensors on and/or in bioreactors. At the same time, there are often stringent requirements relating to minimization of costs. With the multisensor described here it is possible to measure at least three parameters, and therefore when comparison is made with individual sensors it is possible, with the multisensor, to save the spaces required for two sensors. The first measurement arrangement of the multisensor here is configured for an impedance measurement, in particular, by means of impedance spectroscopy and/or of dielectric spectroscopy, and/or for a capacitive measurement, these being particularly preferred measurement methods that are important in many application sectors. Because at least two electrodes of the first measurement arrangement comprise an electrically conductive plastic, or consist thereof, it is possible to realize a particularly low-cost configuration of the multisensor.

Bioreactors, frequently also termed fermenters, generally include a reaction space within which biological or biotechnological procedures can be carried out on laboratory scale. Among these procedures are by way of example the cultivation of cells, of microorganisms or of small plants under defined, preferably optimized, controlled and reproducible conditions. To this end, bioreactors mostly have a plurality of connections by way of which primary and secondary substances, and also various instruments, for example, sensors, can be introduced into the reaction space, or by way of which, for example, fluid lines, in particular, gas lines, for example, gas-supply or gas-discharge lines, can be connected. Bioreactors moreover generally have a stirrer system whose stirrer shaft can be rotated by a drive, thus likewise mostly rotating a stirrer element connected in rotationally rigid manner to the stirrer shaft, and thus bringing about mixing of the substances present in the reaction space. It is also possible to arrange two or more stirrer elements, mostly axially separated, on the stirrer shaft and connected thereto. The stirrer element(s) can also be configured together with the stirrer shaft as a one-piece element. Bioreactors can have various geometries. Dimensionally stable bioreactors can, by way of example, have a cross section, which is preferably a cross section in a plane that is horizontal during operation, and which in essence is circular, oval, triangular, rectangular, square, trapezoidal, or polygonal, or which has a freely selected shape. Flexible bioreactors, can by way of example, be configured as bags and can optionally have dimensionally stable connection equipment.

In both the cell-cultivation application sector and the microbiological application sector, preference is given to the use of bioreactors in, preferably parallel, bioreactor systems. Parallel bioreactor systems are described by way of example in DE 10 2011 054 363.5 or DE 10 2011 054 365.1. Within such a bioreactor system it is possible to achieve parallel operation, and high-precision control, of a plurality of bioreactors. It is also possible here, with small operating volumes in the individual bioreactors, to carry out high-throughput experiments with good reproducibility and scalability. The expression small operating volumes here in particular applies to bioreactors with a size of up to 2000 ml, for example with a total reaction space volume of about 350 ml, with an operating volume of about 60 to about 250 ml.

In the cell-culture application sector, such parallel bioreactor systems are used by way of example for series of experiments based on statistical planning methods (Design of Experiments DoE) for process optimization, for process development, and also for research and development, for example with the aim of culturing various cell lines, such as Chinese hamster ovary (CHO), hybridoma or NSO cell lines. For the purposes of the present text, the expression “cell culture” means, in particular, the culture of animal or plant cells in a nutrient medium outside of the organism. This expression also covers the culture of (human) stem cells, and these cells can likewise be cultured (shaken or stirred) in bioreactors.

In the microbiology application sector, parallel bioreactor systems are likewise used for series of experiments based on statistical planning methods (Design of Experiments DoE) for process optimization, for process development, and also for research and development, for example with the aim of culturing various microorganisms, in particular, bacteria or fungi, e.g., yeasts.

Because laboratory space is mostly limited, it is desirable to achieve low space requirements here, in particular, low standing-space requirements, both for bioreactor systems and for the actual bioreactors.

Bioreactors used in laboratories are often configured from glass and/or metal, in particular from stainless steel, because the bioreactors have to be sterilized between different uses, and this is preferably achieved by sterilization with superheated steam in an autoclave. The sterilization and cleaning of reusable bioreactors is complicated: the sterilization and cleaning procedure can be subject to validation, and its conduct must be documented in detail for each individual bioreactor. Residues in a bioreactor that has not been fully sterilized can distort the results of a subsequent procedure, or render these useless, and can interfere with the conduct of a subsequent procedure. The sterilization procedure can moreover cause stressing of, and sometimes damage to, individual constituents or materials of the bioreactors.

An alternative to reusable bioreactors is provided by disposable bioreactors, which are used to carry out only one biological or biotechnological procedure, with subsequent disposal. The provision, for each procedure, of a new disposable bioreactor, preferably sterilized during the production process, can reduce the risk of (cross)contamination, and at the same time there is no longer any cost for carrying out and documenting correct cleaning and sterilization of a previously used bioreactor. Disposable bioreactors are often configured as flexible containers, for example, as bags or as containers with flexible walls at least in some sections, or are configured as dimensionally stable disposable reactors.

Dimensionally stable disposable bioreactors are often still relatively expensive and designed for pharmaceutical process development and pharmaceutical production processes. They are, in particular, used for cell-culture procedures and are accordingly also, in particular, designed for, and appropriate for, such cell-culture procedures. However, requirements for uses in microbiology are often different, not only in respect of prices achievable in the market but also in respect of suitable design and of materials that can be used for compliance with requirements which are more stringent by several orders of magnitude in respect of technical parameters relating to procedures, examples being mixing time, energy supply and gas exchange.

The invention is, therefore, also based inter alia on the discovery that the advantages of the multisensor described here are particularly valuable specifically in the use with disposable bioreactors for use in cell culture and/or microbiology, because the integration of at least three measurement arrangements in one multisensor here firstly permits considerable reduction of the space required for the measurement of various parameters, and at the same time the use of electrically conductive plastic can achieve very low-cost design. Use of a multisensor also permits reduction of the complexity of the connector materials, for example, because cables and/or other connector materials are required only for one multisensor, instead of three or more individual sensors.

The multisensor can moreover preferably measure the parameters during the procedure, in particular, without the requirement for sampling in which fluids have to be taken from the reaction space and analyzed outside of the reaction space.

The multisensor can have a primary linear-dimensional direction along a longitudinal axis, where the linear dimension of the multisensor along the longitudinal axis and/or in primary linear-dimensional direction is preferably several times greater than a linear dimension orthogonally to the longitudinal axis and/or primary linear-dimensional direction. The multisensor can, by way of example, be configured in the shape of a rod and/or cylinder. The cross section of the multisensor orthogonally to the longitudinal axis and/or primary linear-dimensional direction can moreover be configured to be circular, oval, or polygonal. The multisensor can preferably have a first end and a second end opposite to the first end.

The at least two electrodes of the first measurement arrangement are preferably arranged at a surface of the multisensor and/or arranged in a manner such that during correct use in a bioreactor they can come into contact with fluids located in the reaction space of the bioreactor.

In a preferred embodiment, the multisensor comprises an evaluation unit and/or an interface to an, for example, external evaluation unit, where the evaluation unit, in particular, the evaluation unit of the multisensor, and/or an external evaluation unit is configured, on the basis of an impedance measurement, to derive data concerning biomass situated in the bioreactor, in particular, data concerning cell number and/or cell size and/or cell viability.

Data concerning biomass located in the bioreactor are particularly important for procedures in cell culture and/or in microbiology, and therefore the integration, into the multisensor, of an impedance measurement suitable for this purpose can be particularly advantageous here.

An impedance measurement between intracellular and extracellular fluids can, by way of example, serve for the determination of cell size, in particular, of average cell size. An impedance measurement between cell membranes and fluid can, by way of example, serve for the determination of the number of living cells and/or of cell viability. An impedance measurement between intracellular and extracellular fluids and cell membranes using various frequencies can, by way of example, serve for the determination of cell viability. The impedance measurement is, by way of example, also used as bioelectrical impedance analysis for the determination of the body composition of humans and of other organisms, for example, in body-fat meters.

A capacitive measurement and/or fill-level measurement and/or foam measurement is also particularly important for procedures in cell culture and/or in microbiology, and integration thereof into the multisensor can therefore be particularly preferred. The capacitive measurement and/or fill-level measurement and/or foam measurement is by way of example also used in the CY8CKIT-022 CapSense® Liquid Level Sensing Shield from Cypress (http://www.cypress.com/documentation/development-kitsboards/cy8ckit-022-capsense. liquid-level-sensing-shield).

It is moreover preferable that a second of the three measurement arrangements is configured to carry out an impedance measurement and/or a capacitive measurement and/or a fill-level measurement and/or a foam measurement.

Another preferred embodiment provides that the second measurement arrangement has at least two electrodes which comprise an electrically conductive plastic, or consist thereof.

The at least two electrodes of the first, and/or the at least two electrodes of the second, measurement arrangement can, by way of example, be arranged underneath the surface of the multisensor and/or arranged in a manner such that during correct use in a bioreactor they do not come into direct contact with fluids located in the reaction space of the bioreactor.

A feature of a preferred further development is that a third of the three measurement arrangements is configured to carry out a temperature measurement. It is further preferable that the third measurement arrangement has at least one measurement element for temperature measurement. The measurement element for temperature measurement can by way of example be configured as resistance thermometer.

The first and/or the second and/or the third measurement arrangement can preferably have two or more electrodes which consist entirely or to some extent of electrically conductive plastic or comprise electrically conductive plastic. A conductive plastic can, by way of example, be a plastic with a conductive additive or an intrinsically conductive plastic. Conductive plastics used can in particular be polymers such as polypropylene with a conductive additive (e.g., conductive carbon black, carbon fibers, carbon nanotubes, metal powders or metal fibers, or low-melting-point alloys) or intrinsically conductive polymers (e.g., polyaniline, polythiophene, or polypyrrole).

A preferred embodiment provides that the first and/or the second and/or the third measurement arrangement has/have one, two, or more insulation sections which consist entirely or to some extent of electrically nonconductive and/or insulating plastic or comprise electrically nonconductive and/or insulating plastic. The insulation sections can be arranged between and/or alongside electrodes. The insulation sections can also be arranged on and/or under electrodes, for example, with the aim of preventing direct contact of the electrodes with the fluids surrounding the multisensor, and of forming a protective external surface.

Electrically nonconductive and/or insulating plastic used can in particular be polyolefin, e.g., polypropylene, polyethylene, or a blend of the two.

It is preferable that the electrodes and the insulation sections comprise the same plastic, thus assisting coherent bonding. It is particularly preferable that an electrically conductive polypropylene is used for the electrode and that an electrically nonconductive and/or insulating polypropylene is used for the insulation section.

It is preferable to select materials and/or a combination of materials that meet(s) the requirements of the United States Pharmacopeia (USP) class VI. In particular, it is preferable that the electrically conductive plastic and/or the electrically nonconductive and/or insulating plastic meets the requirements of the United States Pharmacopeia (USP) class VI.

It is further preferable that the first and/or the second and/or the third measurement arrangement has/have been entirely or to some extent produced by molding. A preferred embodiment provides that one, two, or more electrodes of the first and/or of the second and/or of the third measurement arrangement has/have been produced by molding. It is further preferable that one, two, or more insulation sections of the first and/or of the second and/or of the third measurement arrangement has/have been produced by molding. It is also preferable that one, two, or more electrodes of the first and/or of the second and/or of the third measurement arrangement and one, two, or more insulation sections of the first and/or of the second measurement arrangement have been produced by molding.

It is, in particular, preferable that the first and/or the second and/or the third measurement arrangement has been produced entirely or to some extent by injection molding and/or that the first and/or the second and/or the third measurement arrangement has been produced entirely or to some extent by multicomponent injection molding. A preferred embodiment provides that one, two, or more electrodes of the first and/or of the second and/or of the third measurement arrangement have been produced by injection molding. It is further preferable that one, two, or more insulation sections of the first and/or of the second and/or of the third measurement arrangement has/have been produced by injection molding. It is also preferably possible that one, two, or more electrodes of the first and/or of the second and/or of the third measurement arrangement and one, two, or more insulation sections of the first and/or of the second measurement arrangement has/have been produced by multicomponent injection molding.

It is, in particular, preferable that the first and/or the second and/or the third measurement arrangement has/have been produced entirely or to some extent by additive manufacture. A preferred embodiment provides that one, two, or more electrodes of the first and/or of the second and/or of the third measurement arrangement has/have been produced by additive manufacture. It is moreover preferable that one, two, or more insulation sections of the first and/or of the second and/or of the third measurement arrangement has/have been produced by additive manufacture. It is also preferably possible that one, two, or more electrodes of the first and/or of the second and/or of the third measurement arrangement and one, two, or more insulation sections of the first and/or of the second measurement arrangement have been produced by additive manufacture.

The word “molding” here, in particular, means manufacturing processes where a shapeless substance is used to produce a solid body which has a geometrically defined shape.

Examples of molding processes are injection molding and/or multicomponent injection molding. Injection molding and/or multicomponent injection molding can, by way of example, also comprise overmolding and/or sequential overmolding and/or insert injection molding and/or outsert injection molding and/or hybrid injection molding.

The meaning of the phrase “multicomponent injection molding” here, in particular, includes two-component injection molding. Multicomponent injection molding is, in particular, the production of injection moldings made of two or more different plastics or materials, and can be used in composite injection molding and/or in assembly injection molding and/or in sandwich injection molding. A multicomponent injection molding process can use only injection mold, or two or more injection molds.

A multicomponent injection molding process can be implemented in various ways. In the core-back process, after injection and solidification of the first plastics component one or more elements of a mold cavity is/are moved backward to release a further vacant space into which the second plastics component is injected, while the mold however remains closed. In the transfer process, after injection and solidification of the first plastics component, this is inserted, while the mold is open, into a new cavity which has cutouts corresponding to the second plastics component. This can be achieved by using a handling system, where the second cavity can be present in the same mold or indeed in a separate mold and on a second machine. An intermediate solution is provided by transfer within indexing-plate molds, rotary-table molds, or rotating stack molds, where the preform remains either on the core or in the mold cavity of one of the mold halves and, while the mold is open, is transferred into a second cavity by rotation of a region of the mold. Coherence of the preform, which remains on the needle, is an important factor in this solution.

Another example of a molding process is additive manufacturing, which is also termed generative manufacturing or 3D printing. An example of additive manufacturing with use of plastics is provided by the FFF (fused filament fabrication) process, or else by FDM (fused deposition modeling) processes.

The multisensor can also comprise one or more further measurement arrangements. This/these further one or more further measurement arrangements can be configured entirely or to some extent in the same manner as the first and/or the second measurement arrangement and/or the third measurement arrangement, or entirely or to some extent differently.

Production by the injection-molding process and/or by the multicomponent injection-molding process also has the advantage, alongside the advantage of low-cost production, that the elements can be manufactured in one piece and/or integrally, thus permitting avoidance or reduction of joints and/or interstices and/or connection points. It is thus also possible, by way of example, to reduce or avoid the use of adhesive, thus also permitting further reduction of the risk of contamination of the reaction space of a bioreactor.

The multisensor is preferably configured as disposable multisensor. A particular feature of a disposable multisensor is that it is intended for use on a single occasion. To this end, the configuration of the multisensor can be such that after use on a single occasion it is no longer suitable for further use. This can, by way of example, be achieved in that the multisensor is configured entirely or to some extent from materials which do not remain undamaged after a sterilization procedure required for reuse, for example because the temperatures arising during sterilization result in destruction or deformation of all, or some, of the materials. The multisensor can, by way of example, also comprise warnings and/or usage information which exclude multiple use. The mechanical and/or electrical connection can also have been designed in a manner that permits use only on a single occasion.

In another possible variant, the sensor elements located (during operation) in the bioreactor are designed as disposable units (encapsulating electronic systems), but the measurement-electronics system is designed as reusable unit. It is preferable that during the experiment the measurement-electronics system is introduced into the encapsulating sensor unit and held in place, and after the experiment it is transferred into a new disposable vessel with another encapsulating sensor unit, or is stored.

The multisensor can be configured as separate element which, by way of example, can be introduced into a vessel and/or a bioreactor and/or connected thereto and preferably can in turn be separated therefrom and/or removed therefrom.

The multisensor may be configured as a one-piece multisensor or comprise two or more modules connected releasably or non-releasably to one another. A one-piece configuration of the multisensor can preferably be obtained by molding.

The multisensor can also comprise two or more modules, where a module, by way of example, can comprise a measurement arrangement. A module can also comprise two or more measurement arrangements. A module can also comprise one, two, or more parts of a measurement arrangement. Two or more modules can have been connected to one another, for example, by way of a plug connection. The connection can be configured to be releasable or non-releasable, in particular, not releasable without destruction. The connections between different modules can be differently configured. The various modules can respectively be obtained by molding.

The two or more modules can also be configured as a main module and one or more extension modules. It is preferable that the main module comprises a connector head (described in more detail at a later stage below) and/or an evaluation unit and/or an interface to an evaluation unit and/or comprises one or more further elements of the multisensor. An extension module preferably comprises one, two or more measurement arrangements or parts thereof.

A modular structure of the multisensor has inter alia the advantage that, at low cost, it is possible to produce various multisensors, for example, with different measurement arrangements, and that it is thus possible to respond flexibly to customer requirements.

A feature of a preferred further development is that the multisensor comprises a connector head which can be secured on connection equipment of the bioreactor. The connector head can, by way of example, have a screw thread, in particular, an internal screw thread and/or an external screw thread, intended for interaction with a corresponding screw thread of the connection equipment of the bioreactor. It is preferable that the connector head is arranged at a first end of the multisensor. The connector head can moreover have an interface, in particular, an interface to an evaluation unit, in particular, to an external evaluation unit. The interface can preferably be configured for an electrical and/or communication connection.

It is preferable that the multisensor comprises one or more further measurement arrangements, in particular, for the measurement of further parameters, for example, pH and/or dissolved oxygen and/or carbon dioxide content and/or feedstock/product, or concentrations of metabolite, for example: glucose, glutamate, glutamine, ammonium, etc. One or more further measurement arrangements can preferably be arranged at a second end of the multisensor. The one or more further measurement arrangements can preferably have one or more electrodes and/or can comprise electrically conductive plastic and/or electrically nonconductive and/or insulating plastic, or consist of one or more of such materials. Integration of more than three measurement arrangements in a multisensor has the advantage of further reduction of space requirement. With a low-cost design of the multisensor it is moreover possible to eliminate costs for the provision of further individual sensors.

Other advantageous variant embodiments of the device of the invention are obtained by combining the preferred features mentioned here.

In another aspect of the invention, the object mentioned in the introduction is achieved via a bioreactor for use in cell culture and/or in microbiology comprising a multisensor described above.

The bioreactor is preferably configured as disposable bioreactor. A particular feature of a disposable bioreactor is that it is intended for use on a single occasion. To this end, the configuration of the bioreactor can be such that after use on a single occasion it is no longer suitable for further use. This can, by way of example, be achieved in that the bioreactor is configured entirely or to some extent from materials which do not remain undamaged after a sterilization procedure required for reuse, for example, because the temperatures arising during stabilization result in destruction or deformation of all, or some, of the materials. The bioreactor can, by way of example, also comprise warnings and/or usage information which exclude multiple use.

In another aspect of the invention, the object mentioned in the introduction is achieved via a process for the production of a multisensor described above, where the process comprises integration of at least three measurement arrangements into a multisensor, where a first of the three measurement arrangements is configured to carry out an impedance measurement and/or capacitive measurement, and where the first measurement arrangement has at least two electrodes which comprise an electrically conductive plastic or consist thereof.

The process for the production of a multisensor described above further preferably comprises:
molding of the first and/or of the second and/or of the third measurement arrangement in its/their entirety or to some extent; and/or
molding of one, two, or more electrodes of the first and/or of the second and/or of the third measurement arrangement entirely or to some extent from electrically conductive plastic, in particular, electrically conductive polypropylene; and/or
molding of one, two, or more insulation sections of the first and/or of the second and/or of the third measurement arrangement entirely or to some extent entirely or to some extent from electrically nonconductive and/or insulating plastic; and/or
molding of one, two, or more electrodes of the first and/or of the second and/or of the third measurement arrangement and of one, two, or more insulation sections of the first measurement arrangement.

The process for the production of a multisensor described above comprises, in particular:
injection molding of the first and/or of the second and/or of the third measurement arrangement entirely or to some extent, and/or multicomponent injection molding of the first and/or of the second and/or of the third measurement arrangement entirely or to some extent; and/or
injection molding of one, two, or more electrodes of the first and/or of the second and/or of the third measurement arrangement entirely or to some extent from electrically conductive plastic, in particular, from electrically conductive polypropylene; and/or
injection molding of one, two, or more insulation sections of the first and/or of the second and/or of the third measurement arrangement entirely or to some extent entirely or to some extent from electrically nonconductive and/or insulating plastic; and/or
multicomponent injection molding of one, two, or more electrodes of the first and/or of the second and/or of the third measurement arrangement and of one, two, or more insulation sections of the first measurement arrangement;
additive manufacturing of the first and/or of the second and/or of the third measurement arrangement entirely or to some extent; and/or
additive manufacturing of one, two, or more electrodes of the first and/or of the second and/or of the third measurement arrangement entirely or to some extent from electrically conductive plastic, in particular, from electrically conductive polypropylene; and/or
additive manufacturing of one, two, or more insulation sections of the first and/or of the second and/or of the third measurement arrangement entirely or to some extent entirely or to some extent from electrically nonconductive and/or insulating plastic; and/or
additive manufacturing of one, two, or more electrodes of the first and/or of the second and/or of the third measurement arrangement, and of one, two, or more insulation sections of the first measurement arrangement.

In another aspect of the invention, the object mentioned in the introduction is achieved via the use of a multisensor described above for the measurement of at least three parameters in a bioreactor for use in cell culture and/or in microbiology.

In another aspect of the invention, the object mentioned in the introduction is achieved via a process for the measurement of at least three parameters in a bioreactor for use in cell culture and/or in microbiology, where the process comprises:
provision of a multisensor described above,
carrying out an impedance measurement and/or capacitive measurement by using a first of the three measurement arrangements,
carrying out two further measurements by using the second and third of the three measurement arrangements.

In respect of the advantages, variant embodiments and design details of the further aspects of the invention, and advanced forms thereof, reference is made to the preceding description relating to the corresponding features of the multisensor.

The processes described here for the production of a multisensor described above, and for the measurement of at least three parameters in a bioreactor for use in cell culture and/or in microbiology, and also respective advanced forms thereof, in particular comprise features and, respectively, process steps that make them suitable for use for a, and/or with a, multisensor described here and with advanced forms thereof.

### BRIEF DESCRIPTION OF THE DRAWINGS

Preferred embodiments of the invention are described by way of example with reference to the attached figures.

FIG. 1 shows a three-dimensional depiction of a multisensor;

FIG. 2 shows a three-dimensional depiction of a part of a multisensor with a first measurement arrangement;

FIG. 3 shows a three-dimensional depiction of a part of a multisensor with a section of a second measurement arrangement; and

FIG. 4 shows a three-dimensional depiction of a disposable bioreactor with a multisensor.

### DETAILED DESCRIPTION OF THE EMBODIMENTS

Elements that are similar or in essence have the same function are denoted in the figures by identical reference signs.

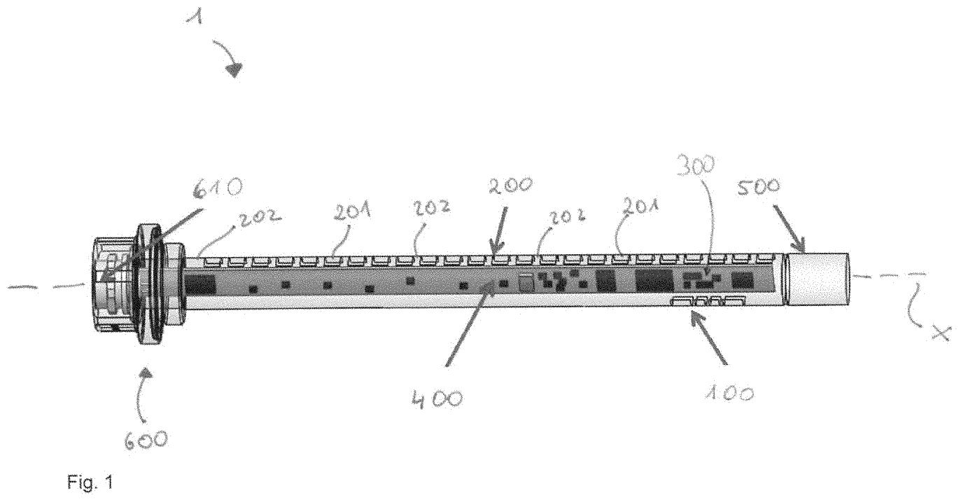

FIG. 1 shows a three-dimensional depiction of one variant of a multisensor 1. The multisensor 1 shown here has a primary linear-dimensional direction along the longitudinal axis X, where the linear dimension of the multisensor 1 along the longitudinal axis X is several times greater than a linear dimension that is orthogonal to the longitudinal axis. The multisensor 1 in the example shown here is configured in the shape of a rod and in essence has the shape of a cylinder. The cross section of the multisensor that is orthogonal to the longitudinal axis and primary linear-dimensional direction is circular.

At a first end of the multisensor 1, there is a connector head 600 arranged, with an interface 610 which is preferably suitable for electrical and/or communication connections.

The multisensor 1 comprises a first measurement arrangement 100, which is configured for an impedance measurement. The multisensor 1 further comprises a second measurement arrangement 200, which is configured to carry out a capacitive measurement and/or a fill-level measurement and/or a foam measurement. The multisensor 1 further comprises a third measurement arrangement 300, which is configured to carry out a temperature measurement.

The multisensor 1 can moreover also comprise further measurement arrangements for the measurement of further parameters, for example, pH and/or dissolved oxygen and/or carbon dioxide content and/or feedstock/product or concentrations of metabolites, for example: glucose, glutamate, glutamine, ammonium, etc.; these can by way of example be arranged at a second end 500, opposite to the first end of the multisensor 1.

The third measurement arrangement 300 is arranged on a component 400 with an integrated electronics system with a microcontroller and with an analog front end. The integrated electronics system of the component 400 can serve as evaluation unit, optionally also together with an external evaluation unit connected by way of the interface arranged in the connector head 600.

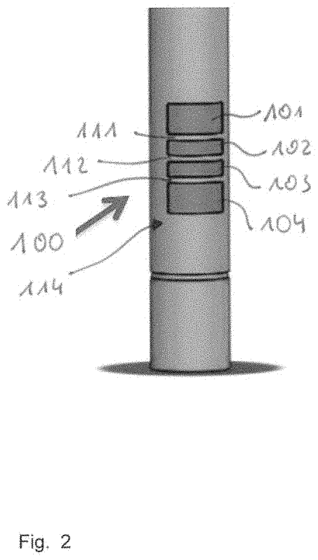

FIG. 2 is an enlarged depiction of a part of a possible variant of a multisensor with a first measurement arrangement 100. The first measurement arrangement 100 preferably comprises four electrodes 101, 102, 103, 104, which respectively are configured from electrically conductive plastic, or comprise electrically conductive plastic. The electrodes 101, 102, 103, 104 are separated and/or surrounded by insulation sections 111, 112, 113, 114, which consist of electrically nonconductive and/or insulating plastic or comprise same. The first measurement arrangement 100 is configured to carry out an impedance measurement. The electrodes 101, 102, 103, 104 are separated from one another by equal distances in the primary linear-dimensional direction of the multisensor 1. The electrodes 101 and 104 have a larger linear dimension in the primary linear-dimensional direction of the multisensor 1 than the two electrodes 102 and 103. The electrodes 101, 102, 103, 104 are arranged at a surface of the multisensor 1 and are arranged in a manner such that, during correct use in a bioreactor, they come into contact with fluids located in the reaction space of the bioreactor.

Preference is given to provision of an evaluation unit of the multisensor 1 and/or of an interface 600 of the multisensor 1 to an evaluation unit, where this evaluation unit is configured, on the basis of an impedance measurement, to derive data concerning biomass situated in the bioreactor, in particular, data concerning cell number and/or cell size and/or cell viability.

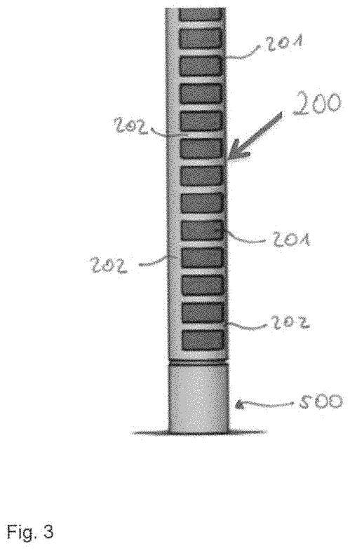

FIG. 3 is an enlarged depiction of a part of a multisensor with a section of a second measurement arrangement 200. The second measurement arrangement 200 is configured to carry out a capacitive measurement and/or a fill-level measurement and/or a foam measurement. The second measurement arrangement 200 moreover has a plurality of electrodes 201, which, in the example depicted here, are separated from one another by equal distances in the primary linear-dimensional direction of the multisensor 1 and comprise an electrically conductive plastic, or consist thereof. Again, these electrodes 201 are separated by insulation sections 202 and/or surrounded by insulation sections 202, where the insulation sections 202 consist of electrically nonconductive and/or insulating plastic, or comprise same. The resolution of the fill-level measurement and/or foam measurement can be influenced by way of the arrangement of the electrodes of the second measurement arrangement 200, in particular, the separation along the longitudinal axis X. The electrodes 201 are arranged beneath the surface of the multisensor and arranged in a manner such that, during correct use in a bioreactor, they do not come into direct contact with fluids located in the reaction space of the bioreactor. To this end, there are preferably also insulation sections configured on the electrodes 201, with the aim of preventing direct contact of the electrodes 201 with the fluids surrounding the multisensor 1, and of forming a protective external surface.

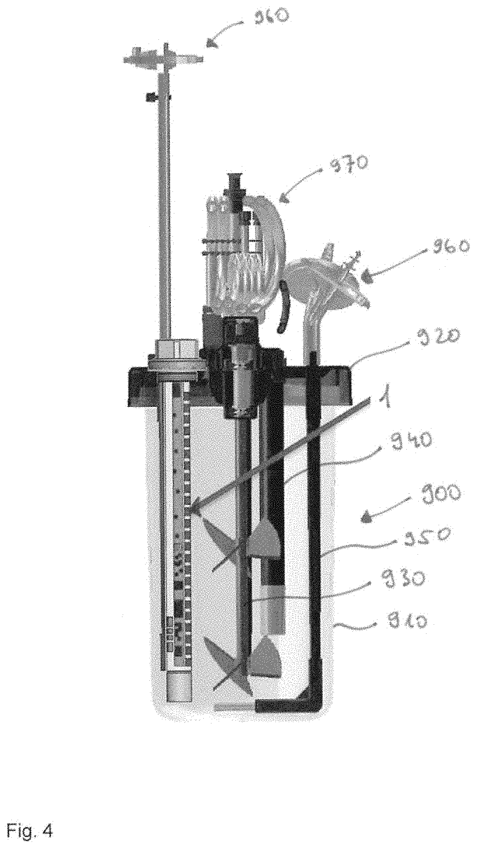

In FIG. 4, the multisensor 1 can be seen arranged in a disposable bioreactor 900. The disposable bioreactor 900 comprises a cover plate 920, a dimensionally stable container 910 and a stirrer unit 930. The cover plate 920 and the container 910 enclose a reaction space. The cover plate 920 has, facing toward the reaction space, an internal side on which a plurality of immersion tubes 940, 950 are arranged, projecting into the reaction space. On an external side of the cover plate 920, facing away from the reaction space, the arrangement has a plurality of connections on which flexible tubes and connection materials 970 and sterile filters 960 are arranged.

When installed in the disposable bioreactor 900, the multisensor 1 is in essence arranged in vertical orientation, and therefore the connector head 600 of the multisensor 1 is arranged at the cover plate 920 of the disposable bioreactor 900, and the multisensor 1 projects along its primary linear-dimensional direction therefrom into the reaction space of the disposable bioreactor 900.

The stirrer unit 930 comprises a stirrer shaft 310 with an axis of rotation and with two stirrer elements configured here with blades inclined by 45°, for example, in the form of pitch blade impeller. Alternatively, it is also possible, by way of example, to use a Rushton impeller as stirrer element. The stirrer elements have been secured in rotationally rigid manner on the stirrer shaft, so that when the stirrer shaft rotates the stirrer elements rotate concomitantly.

The cover plate 920 and the container 910 can, by way of example, be configured from polyamide, or can comprise polyamide, and can have been bonded non-releasably to one another by means of ultrasound welding. The stirrer unit 930, in particular, the stirrer shaft and/or the stirrer elements, can, by way of example, be configured from polystyrene, or can comprise polystyrene.

Flexible tubes and connection materials 970 which used with the disposable bioreactor 900 and which can come into contact with reaction media, are preferably configured from materials certified in accordance with United States Pharmacopeia (USP) class VI, for example, polystyrene, polycarbonate, polyamide, or silicone. The flexible tubes to be used are preferably flexible tubes made of thermoplastic elastomers.

The use of a multisensor 1 in the disposable bioreactor 900 permits use of one connection on the cover plate 920 for the measurement of three (or more) parameters. As can be seen by way of example in FIG. 4, space on the cover plate is limited, but at the same time the number of elements requiring connection here is large. Integration of three sensors into a multisensor is, therefore, especially advantageous, in particular, when the first measurement arrangement is suitable for an impedance measurement and/or for a capacitive measurement.

The use of electrically conductive plastic in the electrodes moreover permits achievement of low-cost design for the multisensor, and this, in particular, also permits configuration thereof as disposable multisensor. Access to further application sectors can thus be achieved.

1.-18. canceled

19. A multisensor for a bioreactor for use in cell culture or in microbiology, the multisensor comprising:
at least three measurement arrangements configured to measure at least three parameters, wherein a first of the three measurement arrangements is adapted to carry out an impedance measurement or a capacitive measurement, and the first measurement arrangement includes at least two electrodes comprising an electrically conductive plastic.

20. The multisensor as claimed in claim 19, further comprising:
an evaluation unit or an interface to the evaluation unit, wherein the evaluation unit is configured to measure impedance via the first measurement arrangement to derive data concerning a biomass situated in the bioreactor.

21. The multisensor as claimed in claim 20, wherein the data concerning the biomass situated in the bioreactor includes cell number, cell size, or cell viability.

22. The multisensor as claimed in claim 19, wherein a second measurement arrangement of the three measurement arrangements is configured to carry out an impedance measurement, a capacitive measurement, a fill-level measurement, or a foam measurement.

23. The multisensor as claimed in claim 22, wherein the second measurement arrangement includes at least two electrodes comprising an electrically conductive plastic.

24. The multisensor as claimed in claim 19, wherein a third measurement arrangement of the three measurement arrangements is configured to carry out a temperature measurement.

25. The multisensor as claimed in claim 19, wherein the first measurement arrangement, a second measurement arrangement, or a third measurement arrangement of the three measurement arrangements comprises two or more insulation sections entirely or partially comprising electrically nonconductive or insulating plastic.

26. The multisensor as claimed in claim 19, wherein the first measurement arrangement, a second measurement arrangement, or a third measurement arrangement of the three measurement arrangements are entirely or partially produced by molding.

27. The multisensor as claimed in claim 26, wherein:
the first measurement arrangement, the second measurement arrangement, or the third measurement arrangement of the three measurement arrangements comprises at least one electrode produced by molding;
the first measurement arrangement, the second measurement arrangement, or the third measurement arrangement of the three measurement arrangements comprises at least one insulation section produced by molding; or
the first measurement arrangement, the second measurement arrangement, or the third measurement arrangement of the three measurement arrangements comprises at least one electrode and at least one insulation section produced by molding.

28. The multisensor as claimed in claim 19, wherein the multisensor is configured as a disposable multisensor.

29. The multisensor as claimed in claim 19, wherein the multisensor is configured as a one-piece multisensor or comprises two or more modules connected releasably or non-releasably to one another.

30. The multisensor as claimed in claim 19, wherein:
a sensor element of the first measurement arrangement, a second measurement arrangement, or a third measurement arrangement of the three measurement arrangements located in the bioreactor is configured as a disposable unit; and
the multisensory further comprises a measurement-electronics system configured as a reusable unit.

31. The multisensor as claimed in claim 19, further comprising a connector head adapted to be secured to a connection interface of the bioreactor.

32. The multisensor as claimed in claim 19, further comprising one or more further measurement arrangements for the measurement of parameters including pH, dissolved oxygen, carbon dioxide content, feedstock/product, or the concentration of metabolites including glucose, glutamate, glutamine, or ammonium.

33. The use of a multisensor as claimed in claim 19 for the measurement of at least three parameters in a bioreactor for use in cell culture and/or in microbiology.

34. A process for the measurement of at least three parameters in a bioreactor for use in cell culture and/or in microbiology, where the process comprises:
provision of a multisensor as claimed in claim 19;
carrying out an impedance measurement or capacitive measurement by using the first of the three measurement arrangements; and
carrying out two further measurements by using a second measurement arrangement and a third measurement arrangement of the three measurement arrangements.

35. A bioreactor for use in cell culture and/or in microbiology comprising a multisensor, the multisensor further comprising:
at least three measurement arrangements configured to measure at least three parameters, wherein a first of the three measurement arrangements is adapted to carry out an impedance measurement or a capacitive measurement, and the first measurement arrangement includes at least two electrodes comprising an electrically conductive plastic.

36. The bioreactor as claimed in claim 35, where the bioreactor is configured as a disposable bioreactor.

37. A process for the production of a multisensor for a bioreactor for use in cell culture or in microbiology, the multisensor comprising at least a first measurement arrangement, a second measurement arrangement, and a third measurement arrangement configured to measure at least three parameters, wherein the process comprises the step of:
Integrating the at least first, second, and third measurement arrangements into the multisensor, where the first measurement arrangement is adapted to carry out an impedance measurement or a capacitive measurement, and the first measurement arrangement includes at least two electrodes comprising an electrically conductive plastic.

38. The process as claimed in claim 35, further comprising the step of:
molding the first, the second, or the third measurement arrangements in its entirety as an integrated unit;
molding the at least two electrodes of the first measurement arrangement or a one or more of an electrode of the second or the third measurement arrangements entirely or partially from electrically conductive plastic;
molding one or more insulation sections of the first, the second, the third measurement arrangement entirely or partially from electrically nonconductive or insulating plastic; or
molding the at least two electrodes of the first measurement arrangement or a one or more of an electrode of the second or the third measurement arrangement and one or more insulation sections of the first measurement arrangement.
